(19) United States
(12) Patent Application Publication
(10) Pub. No.: US 2022/0183941 A1
(43) Pub. Date: Jun. 16, 2022

(54) SILICONE OIL-FREE ANTIPERSPIRANTS

(71) Applicant: Henkel AG & Co. KGaA, Duesseldorf (DE)

(72) Inventor: Thomas DOERING, Dormagen (DE)

(73) Assignee: Henkel AG & Co. KGaA, Duesseldorf (DE)

(21) Appl. No.: 17/547,045

(22) Filed: Dec. 9, 2021

(30) Foreign Application Priority Data
Dec. 11, 2020 (DE) 102020133123.1

(51) Int. Cl. A61K 8/26; A61K 8/37; A61K 8/86; A61K 8/31; A61Q 15/00

(52) U.S. Cl. CPC A61K 8/26; A61K 8/37; A61K 8/86; A61K 2800/591; A61Q 15/00; A61K 2800/34; A61K 2800/31; A61K 8/31

(57) ABSTRACT

The present disclosure relates to an antiperspirant cosmetic composition that provides good efficacy without silicone oils. Furthermore, the present disclosure relates to a cosmetic method for reducing perspiration and/or body odor caused by perspiration using a composition as contemplated herein. The cosmetic composition used in the cosmetic method is free of silicone oils.

### CROSS-REFERENCE TO RELATED APPLICATION

This application claims priority to German Patent Application No. 102020133123.1, filed Dec. 11, 2020, which is incorporated herein by reference in its entirety.

### TECHNICAL FIELD

The present disclosure relates to an antiperspirant cosmetic composition that provides good efficacy without silicone oils. Furthermore, the present disclosure relates to a cosmetic method for reducing perspiration and/or body odor caused by perspiration using a composition as contemplated herein.

Washing, cleaning and caring for one's own body is a basic human need and modem industry is constantly trying to meet these needs in a variety of ways. Particularly important for daily hygiene is the permanent elimination or at least reduction of body odor and underarm wetness. Numerous special deodorizing or antiperspirant body care products are known in the state of the art, which have been developed for use in body regions with a high density of sweat glands, especially in the armpit area. These are available in a wide variety of dosage forms, for example as powder, in stick form, as aerosol spray, pump spray, liquid and gel roll-on application, cream, gel and as impregnated flexible substrates (deodorants).

### BACKGROUND

State-of-the-art cosmetic antiperspirants contain, in addition to at least one oil or wax and a fragrance component or perfume, at least one antiperspirant compound, for example in the form of aluminum salts and/or aluminum-zirconium salts, in the form of halides and/or hydroxyhalides of aluminum and/or zirconium.

Antiperspirants and deodorants usually contain silicone oils to create a good feeling on the skin. Decamethylcyclopentasiloxane is frequently used. Furthermore, corresponding silicone oils have the advantage that they are volatile so that they do not leave stains or smear films on the user's clothing. In addition, the volatility of these silicone oils enables the release of the antiperspirant aluminum salt, so that it can first develop its effect in the interaction with sweat. However, the volatility of silicone oils causes white stains to be visible on the user's clothing. However, silicone oils are increasingly criticized because of their impact on the environment.

There is therefore a need for antiperspirants and deodorants that do not pollute the environment but at the same time have a satisfactory performance in terms of sweat reduction or reduction of body odor caused by sweat. In addition, the application of the agent to the skin should not stain the user's clothing.

Surprisingly, it was found that an aluminum salt in combination with medium-chain alkanes as well as triester of tricarboxylic acids and PPG-14 butyl ether are particularly effective and provide a particularly good skin feel.

Thus, a first object of the present disclosure is an anhydrous cosmetic composition comprising:
a) from about 5 to about 40% by weight of at least one aluminum salt
b) from about 5 to about 15% by weight of at least one alkyl triester of a C₄₋₈ tricarboxylic acid with a C₁₋₄ alkyl alcohol,
c) from about 5 to about 20% by weight of at least one C₅₋₂₀ alkane and
d) from about 5 to about 20% by weight of PPG-14 butyl ether, the composition being free from volatile silicone oils. Preferably, the composition is free of silicone oils, that is, free of volatile and non-volatile silicone oils.

The cosmetic composition is an anhydrous suspension. Antiperspirant compositions are often offered in dosage forms that feature metal. The presence of water in these cases can lead to corrosion of the dosage form. Therefore, the composition as contemplated herein is anhydrous. Anhydrous in the sense of the present disclosure means that the water content is less than about 0.5% by weight, preferably about 0.2% by weight or less, about 0.1% by weight or less. Particularly preferably, no free water is present in the composition.

This can be presented, for example, as an aerosol spray, roll-on application or deodorant cloth (impregnated flexible substrate).

The composition is free of volatile silicone oils, preferably it is free of any silicone oils. In terms of the present disclosure, this means that the amount of volatile silicone oils or, in a preferred embodiment, any silicone oils does not exceed about 0.1% by weight. The amount of volatile silicone oils or, in a preferred embodiment, any silicone oils is thus in the range of 0 to about 0.1% by weight. If the cosmetic composition as contemplated herein contains volatile silicone oils or, in a preferred embodiment, volatile or non-volatile silicone oils, these have not been actively added to the composition but are present as an impurity of one of the metered components. Corresponding contamination cannot always be completely avoided.

The term “volatile silicone oils” refers to compounds which have a vapor pressure of about 2.66 Pa to about 40,000 Pa at 20° C. and an ambient pressure of 1,013 hPa, preferably from about 10 to about 12,000 hPa, more preferably from about 13 to about 3,000 hPa, from about 15 to about 500 hPa.

### BRIEF SUMMARY

Cosmetic antiperspirant compositions and methods for their use are provided. In an exemplary embodiment, an anhydrous antiperspirant cosmetic composition includes a) from about 5 to about 40% by weight of at least one aluminum salt, and b) from about 5 to about 15% by weight of at least one alkyl triester of a C₄₋₈ tricarboxylic acid with a C₁₋₄ alkyl alcohol. The composition also includes c) from about 5 to about 20% by weight of at least one C₅₋₂₀ alkane and d) from about 5 to about 20% by weight of PPG-14 butyl ether. The composition is free of volatile silicone oils.

A method of reducing body odor with a cosmetic composition is provided in another embodiment. The cosmetic composition is applied to the skin, where the cosmetic composition includes from about 5 to about 40% by weight of at least one aluminum salt, and from about 5 to about 15% by weight of at least one alkyl triester of a C₄₋₈ tricarboxylic acid with a C₁₋₄ alkyl alcohol. The cosmetic composition also includes from about 5 to about 20% by weight of at least one C₅₋₂₀ alkane, and from about 5 to about 20% by weight of PPG-14 butyl ether. The cosmetic composition is free of volatile silicone oils. The cosmetic composition is then left on the skin for at least 1 hour.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

As contemplated herein, the term “antiperspirant” is understood to mean the decrease or reduction of perspiration of the body's sweat glands, or the reduction of body odor caused by sweat. Thus, an antiperspirant cosmetic composition within the meaning of the present disclosure may be an antiperspirant or a deodorant. Preferably, it is an antiperspirant. This is a non-therapeutic product that can be used as part of daily personal hygiene.

Where quantities are given in this application, these are percentages by weight, unless explicitly stated otherwise. Unless otherwise stated, all quantities refer to the total weight of the composition as contemplated herein. Any blowing agents that may have been added do not count as part of the composition as contemplated herein, so all quantities given refer to the total weight of the blowing agent-free composition, unless otherwise stated.

The composition as contemplated herein has at least one aluminum salt. The at least one aluminum salt may be present, for example, in the form of aluminum salts and/or aluminum-zirconium salts, in the form of halides and/or hydroxyhalides. In the context of the present disclosure, the term “halides and/or hydroxyhalides” refers to chlorides, bromides and iodides of aluminum and aluminum-zirconium, as well as compounds of the formulae Al(OH)_{y}X, where X in the above formulae stands for a halide ion. These are especially chlorides.

Preferred antiperspirant cosmetic compositions are therefore exemplified in that they contain at least one aluminum salt with a molar ratio of aluminum to chloride of from about 1.26:1 to about 1.90:1, preferably from about 1.35:1 to about 1.88:1, more preferably from about 1.40:1 to about 1.88:1, in particular from about 1.65:1 to about 1.88:1. The use of aluminum salts with the aforementioned molar ratios of aluminum to chloride has been shown to be particularly beneficial in terms of antiperspirant activity, reduced residue formation, and improved dryness sensation.

Furthermore, it may be preferred as contemplated herein if the aluminum salts used have a high chloride content. Preferred antiperspirant cosmetic compositions are therefore exemplified in that they contain at least one aluminum salt with a chloride content of from about 5% to about 35% by weight, preferably from about 10% to about 30% by weight, more preferably from about 15% to about 25% by weight, from about 17% to about 20% by weight, based on the total weight of the aluminum salt. The use of such salts leads to a high antiperspirant effect as well as to a reduced formation of residues compared to aluminum salts with different, especially lower, chloride contents. The chloride content of the aluminum salts can be determined, for example, by potentiometric titration against a silver nitrate solution as standard.

It has been found to be advantageous in the context of the present disclosure if the antiperspirant cosmetic composition contains aluminum salts which are in activated form, i.e., have been activated with heat, and have a high proportion of polymeric aluminum complexes (hereinafter also referred to as Al complexes) with a low molecular weight. These polymeric Al complexes are formed by dissociation of aluminum salts into Al(III) aqua complexes and subsequent formation of polymeric Al complexes with hydroxide and oxide bridges between aluminum ions. However, polymeric Al complexes with a high molecular weight exhibit a lower antiperspirant effect than polymeric Al complexes with a low molecular weight.

For example, the formation of polymeric Al complexes with low molecular weights can be determined by size exclusion chromatography. For this purpose, a column containing salinized porous silica microspheres with a particle size of about 5 um is used (for example, Agilent® Zorbax® PSM 60-S column, which is commercially available from Agilent®). About 0.02 M HCl is suitable as eluent. Upon analysis of the aluminum salts used as contemplated herein, 5 to 6 peaks are obtained, representing polymeric Al complexes with different molecular weight ranges. Peaks 1 and 2 represent polymeric Al complexes with an average molecular weight of from about 5,000 to about 10,000 g/mol, peak 3 represents polymeric Al complexes with an average molecular weight of from about 2,000 to about 4,000 g/mol, and peak 4 represents polymeric Al complexes with an average molecular weight of from about 500 to about 1,500 g/mol. Finally, peaks 5 and 6, respectively, represent polymeric Al complexes with average molecular weights ranging from about 135 to about 270 g/mol.

The area of the respective peak is directly proportional to the amount of polymeric Al complexes of the respective molecular weight range. Therefore, by comparing the area of one peak with the total area of all peaks obtained, the proportion of polymeric Al complexes of the corresponding molecular weight range in the total amount of polymeric Al complexes formed in the aluminum salt used as contemplated herein can be directly determined.

Peak 4 contains the polymeric Al complexes of low molecular weight that are significant for the antiperspirant effect. Regarding the antiperspirant effect, it is therefore advantageous as contemplated herein if the area fraction of peak area 4 lies within a certain range compared to the total area of all peak areas 1 to 5 or 1 to 6 in the chromatogram. Preferred antiperspirant cosmetic compositions as contemplated herein are therefore exemplified in that they contain at least one aluminum salt with a peak area ratio of peak 4 to the total area of all peaks in the chromatogram of from about 40 to about 80%, preferably from about 42 to about 75%, more preferably from about 45 to about 70%, from about 50 to about 65%. The peak area fraction of peak 4 with respect to the total area of all peaks in the chromatogram was determined by size exclusion chromatography using an Agilent® Zorbax® PSM 60S column with 0.02 M HCl as eluent. The total area of all peaks here refers to the sum of all peaks obtained for the aluminum salt in the chromatogram. The use of such aluminum salts has proven to be particularly advantageous in terms of good antiperspirant efficacy in combination with a silicone oil-free matrix.

At least one aluminum salt is preferably used in specific ranges of amounts. Therefore, as contemplated herein, it is advantageous if the cosmetic composition contains—based on its total weight—from about 8 to about 38% by weight, preferably from about 10 to about 35% by weight, preferably from about 12 to about 35% by weight, from about 15 to about 30% by weight, of at least one aluminum salt. The use of the total amounts of aluminum salt listed above results in excellent antiperspirant performance without causing incompatibilities with other ingredients of the cosmetic composition as contemplated herein or excessive residue formation on textiles.

As a second essential ingredient b), the antiperspirant cosmetic composition as contemplated herein contains at least one alkyl triester of a C₄₋₈ tricarboxylic acid with a C₁₋₄ alkyl alcohol. In particular, the tricarboxylic acid is selected from citric acid, aconitic acid, isocitric acid and oxalsuccinic acid, preferably it is selected from citric acid and isocitric acid. The C₁₋₄ alkyl alcohol is particularly selected from ethanol and propanol, especially preferably propanol. Thus, triethyl or tripropyl esters of citric acid or of isocitric acid are particularly preferred. Triethyl citrate is particularly preferred.

Preferred antiperspirant cosmetic compositions as contemplated herein are exemplified in that they comprise—based on their total weight—from about 5% to about 15% by weight, preferably from about 6% to about 14% by weight, more preferably from about 6% to about 13% by weight, from about 7% to about 12% by weight of at least one alkyl triester of a C₄₋₈ tricarboxylic acid with a C₁₋₄ alkyl alcohol.

As a third essential ingredient c), the antiperspirant cosmetic composition as contemplated herein contains at least one medium-chain alkane having 5 to about 20 carbon atoms. Preferably, the medium-chain alkane has 6 to about 18 C atoms, 9 to about 12 C atoms. For example, if C₉₋₁₂ alkanes are described, the composition is one that has the named alkanes.

Alkanes in the sense of the present disclosure are hydrocarbons which have the chain length (number of C atoms) mentioned. As contemplated herein, these alkanes can be linear, branched or cyclic. Preferably, the medium-chain alkanes are linear or branched.

Preferred antiperspirant cosmetic compositions as contemplated herein are exemplified in that they comprise—based on their total weight—from about 5 to about 20% by weight, preferably from about 6 to about 18% by weight, from about 7 to about 15% by weight, more preferably from about 7 to about 12% by weight of medium-chain alkane. The alkanes make an important contribution to the non-greasy skin feel of the composition as contemplated herein.

As the fourth essential ingredient d), the antiperspirant cosmetic composition as contemplated herein contains PPG-14 butyl ether, i.e., a polyoxypropylene (14) butyl ether. PPG-14 butyl ether is a propylene glycol ether of butyl alcohol, which can be exemplified by the following formula: C₄H₉(OC(CH₃)CH₂)ₙOH, where n is 14. Due to the propylene glycol content, PPG-14 butyl ether is exemplified by a moderately hydrophilic character, which is very suitable for dispersing hydrophilic ingredients of the cosmetic composition. PPG-14 butyl ether can also be used to disperse salts, such as the aluminum salt used in the present disclosure, in the composition.

Preferred antiperspirant cosmetic compositions as contemplated herein are exemplified in that they contain—based on their total weight—from about 5% to about 20% by weight, preferably from about 6% to about 18% by weight, preferably from about 7.0% to about 16% by weight, from about 7.0% to about 15% by weight of PPG-14 butyl ether.

In a preferred embodiment, the present disclosure therefore discloses an anhydrous cosmetic composition comprising:
a) from about 5 to about 40% by weight of at least one activated aluminum sesquichlorohydrate,
b) from about 5 to about 15% by weight of triethyl citrate,
c) from about 5 to about 20% by weight of at least one C₉₋₁₂ alkane and
d) from about 5 to about 20% by weight of PPG-14 butyl ether, the composition being free from volatile silicone oils. Preferably, the composition is free of silicone oils, that is, free of volatile and non-volatile silicone oils.

If the composition as contemplated herein is present as an aerosol spray, it further comprises at least one propellant. In the context of the present disclosure, it is preferred to use certain blowing agents. Advantageous antiperspirant cosmetic compositions as contemplated herein are therefore exemplified in that they contain at least one propellant selected from the group of propane, propene, n-butane, iso-butane, iso-butene, n-pentane, pentene, iso-pentane, iso-pentene, methane, ethane, dimethyl ether, nitrogen, air, oxygen, nitrous oxide, 1,1,1,3-tetrafluoroethane, heptafluoro-n-propane, perfluoroethane, monochlorodifluoromethane, 1,1-difluoroethane, tetrafluoropropenes and mixtures thereof, in particular propane and/or n-butane and/or iso-butane. Furthermore, as contemplated herein, it is also possible to use mixtures of the blowing agents with hydrophilic blowing agents, such as carbon dioxide and/or dimethyl ether.

To ensure sufficient sprayability and complete extraction of the compositions as contemplated herein in this embodiment, the propellant is preferably used in certain quantity ranges. It is therefore advantageous in the context of the present disclosure if the cosmetic composition contains—based on its total weight—from about 40% to about 90% by weight, preferably from about 55% to about 88% by weight, preferably from about 60% to about 86% by weight, from about 70% to about 85% by weight, of at least one propellant, in particular propane and/or n-butane and/or iso-butane.

Pressure-resistant containers for such aerosols can be made of metal (aluminum, tinplate, tin), protected or non-splintering plastic, or glass coated on the outside with plastic. Pressure and breakage resistance, corrosion resistance, ease of filling, as well as aesthetic considerations, handiness, printability, etc., all play a role in their selection. Special internal protective coatings ensure corrosion resistance to the cosmetic composition located in the pressure vessel. Due to the absence of water, however, the corrosiveness of the composition is significantly reduced, so that corresponding protective coatings are only required to a limited extent or not at all. In particular, the valves used have an internally painted valve head, whereby the paint and valve material are compatible with each other. If aluminum valves are used, their valve discs can be coated on the inside with Microflex paint, for example. If tinplate valves are used as contemplated herein, their valve discs can be coated on the inside with PET (polyethylene terephthalate), for example. Coating is not necessary in the compositions as contemplated herein, but it is also not troublesome. The sizes of the aerosol droplets and the respective size distribution can be adjusted for a given spray device via the quantity ratio of propellant to the other ingredients of the cosmetic composition.

However, it may be equally preferred as contemplated herein if the cosmetic composition is sprayed as a propellant-free pump spray or squeeze spray or applied as a roll-on.

The composition as contemplated herein may be in the form of a suspension, for example. To stabilize the suspension, the composition as contemplated herein may contain ingredients that can prevent or reduce sedimentation, i.e., improve the stability of the suspension. One, two, three or more suitable thickeners can be added for this purpose.

Depending on the desired application and form of application, the composition as contemplated herein may comprise further components, in particular excipients. These are preferably those selected from the group of solvents, perfume oils, emulsifiers, solubilizers, cosmetic oils, consistency agents, propellants, pH-adjusting agents, surfactants, chelating agents, skin-cooling active ingredients, skin-caring active ingredients, waxes, preservatives, plant extracts, absorbers and mixtures of these excipients.

The composition as contemplated herein not only has an antiperspirant effect but can also be used to mask odors. To this end, the composition preferably further comprises at least one fragrance. In the context of the present disclosure, “perfume” means substances with a molecular weight of from about 74 to about 300 g/mol, which contain at least one osmophoric group in the molecule and have an odor and/or taste, i.e., which can excite the receptors of the hair cells of the olfactory system. Osmophoric groups are groups covalently bound to the molecular skeleton in the form of hydroxy groups, formyl groups, oxo groups, alkoxycarbonyl groups, nitrile groups, azide groups, etc. In the context of the present disclosure, perfume oils, perfumes or perfume oil components which are liquid at 20° C. and 1013 hPa are understood. In this context, the fragrance is preferably present in the composition as contemplated herein in an amount of from about 0.001% by weight to about 10% by weight, particularly om about 0.005% by weight to about 9% by weight, further preferably from about 0.1% by weight to about 7% by weight, particularly preferably from about 0.2% by weight to about 6% by weight and especially from about 0.2% by weight to about 2% by weight, in each case based on the total weight of the composition as contemplated herein.

To further support the odor-reducing effect of the composition as contemplated herein, in a preferred embodiment the composition as contemplated herein further comprises one or more deodorant active ingredients. As contemplated herein, the use of certain deodorant active ingredients has proven to be particularly advantageous. It is therefore preferred as contemplated herein if the at least one deodorant active ingredient is selected from the group of (i) arylsulfatase inhibitors, β-glucuronidase inhibitors, aminoacylase inhibitors, esterase inhibitors, lipase inhibitors, lipoxigenase inhibitors and cystathionine β-lyase inhibitors; (ii) α-monoalkylglycerol ethers with a branched or linear saturated or unsaturated, optionally hydroxylated C₆₋₂₂ alkyl radical, in particular α-(2-ethylhexyl)glycerol ethers, (iii) alcohols, in particular phenoxyethanol, benzylheptanol, 1,2-hexanediol, 1,2-octanediol, 1,2-decanediol, tropolone and butyloctanoic acid; (iv) germ-inhibiting perfume oils; (v) prebiotically active components; (vi) active ingredients that reduce the number of or inhibit the growth of skin germs from the group of staphylococci, coryne bacilli, anaerococci and micrococci that are involved in odor formation; (vii) active ingredients that reduce the number of or inhibit the growth of skin germs from the group of staphylococci, coryne bacilli, anaerococci and micrococci. (vii) Zinc and silver compounds, in particular zinc phenolsulfonate, zinc ricinoleate, bischloride(-1)-octahydroxy-pentazinc, zinc citrate and silver lactate; (viii) Sorbitan esters and lactones, in particular sorbitan caprylate and glucono-1,5-lactone; (ix) organohalogen compounds, especially triclosan, chlorhexidine and chlorhexidine gluconate; (x) benzalkonium halides, especially benzalkonium chloride and benzethonium chloride; (xi) quaternary ammonium compounds, especially cetylpyridinium chloride; (xii) antimicrobial carbonates, phosphates and sulfates, especially sodium bicarbonate, cocamidopropyl PG-dimonium chloride phosphates, disodium pyrophosphate and soya morpholinium ethosulfates; (xiii) lantibiotics; (xiv) aluminum and aluminum zirconium salts; (xv) bispyridines, especially octenidine; (xvi) antimicrobially active acids, in particular caprylic hydroxamic acid, carnesolic acid, tartaric acid and 2-butyloctanoic acid; (xvii) polyglycerol esters, in particular polyglyceryl-3 caprylates; (xviii) and mixtures thereof, in particular phenoxyethanol and/or caprylylglycol and/or 1,2-hexanediol and/or a-(2-ethylhexyl)glycerol ether and/or 2-butyloctanoic acid. The use of these active ingredients leads not only to a high and long-lasting perfuming and storage stability, but also to a high and long-lasting deodorizing effect.

Another object of the present disclosure is the non-therapeutic use of the composition as contemplated herein for preventing and/or reducing body odor and/or perspiration. Non-therapeutic in the sense of the present disclosure means that the use is in the sense of daily personal hygiene, without a therapeutic approach.

A still further object of the present disclosure is a non-therapeutic cosmetic method for preventing and/or reducing body odor and/or perspiration of the body, wherein a cosmetic composition as previously described is applied to the skin, in particular to the skin of the armpits, and is left on the skin of the armpits for at least 1 hour, preferably for at least 2 hours, preferably for at least 4 hours, in particular for at least 6 hours.

Preferred embodiments of the present disclosure may be combined in any way, regardless of the embodiment or subject matter of the present disclosure. Thus, preferred embodiments described for the composition apply mutatis mutandis to the use and method, and vice versa.

In the following embodiments, the present disclosure is explained in a non-limiting manner.

### 1. Antiperspirant Effectiveness

Formulation V1 (comparative example) represents a commercially available silicone oil-based suspension that can be filled into aerosol cans for application as an aerosol together with a propellant (e.g., propane/butane). For testing the antiperspirant efficacy, the propellant can be neglected, as it does not remain on the skin. Table 1 describes the compositions of the two formulations.

TABLE 1

| | E (present | V1 |
|---|---|---|
| | disclosure) | (comparison) |
| Activated aluminum | 33.3 | 33.3 |
| sesquichlorohydrate (AASCH- | | |
| 3143, Elementis ®) | | |
| Cyclopentasiloxane | — | 43.0 |
| C9-12 alkane (Parafol ® 12-97, | 10.0 | — |
| Sasol ®) | | |
| PPG-14 Butyl Ether (Ucon Fluid | 10.0 | — |
| AP, Dow ®) | | |
| Ethylhexyl palmitate (Tegosoft ® | 26.6 | 7.0 |
| OP, Evonik ®) | | |
| Triethylcitate (Citrofol AI, | 10.0 | — |
| Junbunzlauer) | | |
| Bentone ® 38 V CG | 2.5 | 2.5 |
| Propylene carbonate | 0.9 | 0.9 |
| Perfume | 6.7 | 6.7 |

Formulation V1 and formula E as contemplated herein were applied to defined areas on the back of 28 volunteers (2 μl/cm²) and evenly distributed. The treated skin sites were covered with an occlusive patch for 2 hours. The application of each formulation was repeated daily for 4 consecutive days. The contralateral area in each case served as an untreated control. 72 h after the last application, the treated areas and untreated control sites were covered with moisture-absorbing pads. Subsequently, sweating was induced by the subjects' stay in a sauna at 80° C. The amount of sweat absorbed was determined gravimetrically and placed in percentage relation to the untreated control field. Table 2 shows that the formulation as contemplated herein has a higher weld reduction after 72h compared to V1.

TABLE 2

| | Sweat reduction after 72 h |
|---|---|
| E | −49.3% |
| V1 | −39.9% |

### 2. Application Properties

Aerosol products E and V2 (Table 3) were applied to the axilla by 5 sensory experts. Sensory attributes were rated using a 0-15 scale (see Table 4). The results in Table 4 show that the formulation as contemplated herein leads to an improvement in skin feel in terms of tackiness and oiliness compared to V2 with C₉₋₁₂ alkanes.

TABLE 3

| | E (present | V2 |
|---|---|---|
| | disclosure) | (comparison) |
| Activated aluminum | 33.3 | 33.3 |
| sesquichlorohydrate (AASCH- | | |
| 3143, Elementis ®) | | |
| C9-12 alkane (Parafol ® 12-97, | 10.0 | — |
| Sasol ®) | | |
| PPG-14 Butyl Ether (Ucon Fluid | 10.0 | 10.0 |
| AP, Dow ®) | | |
| Ethylhexyl palmitate (Tegosoft ® | 26.6 | 36.6 |
| OP, Evonik ®) | | |
| Triethylcitate (Citrofol AI, | 10.0 | 10.0 |
| Junbunzlauer) | | |
| Bentone ® 38 V CG | 2.5 | 2.5 |
| Propylene carbonate | 0.9 | 0.9 |
| Perfume | 6.7 | 6.7 |

The formulations were filled in aerosol cans in a weight ratio of 1:5.7 with the blowing agent propane/butane (15/85).

TABLE 4

| | White residue | Stickiness | Oiliness |
|---|---|---|---|
| E | 1.5 | 5.5 | 8.0 |
| V2 | 2.0 | 9.0 | 11.5 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An anhydrous antiperspirant cosmetic composition comprising, based on a total weight of the cosmetic composition:
a) from about 5 to about 40% by weight of at least one aluminum salt,
b) from about 5 to about 15% by weight of at least one alkyl triester of a C₄₋₈ tricarboxylic acid with a C₁₋₄ alkyl alcohol,
c) from about 5 to about 20% by weight of at least one C₅₋₂₀ alkane and
d) from about 5 to about 20% by weight of PPG-14 butyl ether, wherein the composition is free from volatile silicone oils.

2. The composition of claim 1, wherein the at least one aluminum salt is an activated aluminum sesquichlorohydrate.

3. The composition of claim 2, wherein the at least one aluminum salt has a molar ratio of aluminum to chlorine of from about 1.26:1 to about 1.90:1.

4. The composition of claim 2, wherein for the at least one aluminum salt the peak 4 fraction of a size exclusion chromatography is from about 40% to about 80%, wherein the size exclusion chromatography uses a column containing salinized porous silica microspheres with a particle size of 5 μm and a 0.02 M HCl eluent.

5. The composition according to claim 1, wherein the composition comprises silicone oil as an impurity in a proportion of about 0.5% by weight or less, based on the total weight of the composition.

6. The composition according to claim 1, wherein the at least one alkyl triester of a C₄₋₈ tricarboxylic acid with a C₁₋₄ alkyl alcohol is selected from the group of triethyl esters of citric acid, tripropyl esters of citric acid, isocitric acid, and combinations thereof

7. The composition according to claim 1, wherein the at least one C₅₋₂₀ alkane comprises a C₆₋₁₈ alkane.

8. The composition according to claim 2, wherein the at least one aluminum salt has a molar ratio of aluminum to chlorine of from about 1.65:1 to about 1.88:1

9. The composition according to claim 1, comprising, based on the total weight of the composition:
a) from about 5 to about 40% by weight of at least one activated aluminum sesquihydrate,
b) from about 5 to about 15% by weight of triethyl citrate,
c) from about 5 to about 20% by weight of at least one C₉₋₁₂ alkane and
d) from about 5 to about 20% by weight of PPG-14 butyl ether, and wherein the composition is free from volatile silicone oils.

10. A non-therapeutic cosmetic method for preventing and/or reducing body odor and/or perspiration of the body, comprising the steps of:
applying a cosmetic composition to the skin, wherein the cosmetic composition comprises, based on a total weight of the cosmetic composition;
a) from about 5 to about 40% by weight of at least one aluminum salt;
b) from about 5 to about 15% by weight of at least one alkyl triester of a C₄₋₈ tricarboxylic acid with a C₁₋₄ alkyl alcohol;
c) from about 5 to about 20% by weight of at least one C₅₋₂₀ alkane; and
d) from about 5 to about 20% by weight of PPG-14 butyl ether;
and wherein the cosmetic composition is free of volatile silicone oils; and
leaving the cosmetic composition on the skin for at least 1 hour.

11. The composition according to claim 1, wherein the at least one aluminum salt has a molar ratio of aluminum to chlorine of from about 1.35:1 to about 1.88:1.

12. The composition according to claim 1, wherein the at least one aluminum salt has a molar ratio of aluminum to chlorine of from about 1.40:1 to about 1.88:1.

13. The composition according to claim 1, wherein the composition comprises silicone oil as an impurity in a proportion of from 0 to about 0.1% by weight, based on the total weight of the composition.

14. The composition according to claim 1, wherein the at least one C₅₋₂₀ alkane comprises a C₉₋₁₂ alkane.

15. The composition according to claim 1, wherein the at least one alkyl triester of a C₄₋₈ tricarboxylic acid with a C₁₋₄ alkyl alcohol comprises a triethyl ester of citric acid.

16. The composition according to claim 1, wherein the at least one alkyl triester of a C₄₋₈ tricarboxylic acid with a C₁₋₄ alkyl alcohol comprises a tripropyl ester of citric acid.

17. The composition according to claim 1, wherein the at least one alkyl triester of a C₄₋₈ tricarboxylic acid with a C₁₋₄ alkyl alcohol comprises isocitric acid.

18. The composition of claim 2, wherein for the at least one aluminum salt the peak 4 fraction of a size exclusion chromatography is from about 50% to about 65%, wherein the size exclusion chromatography uses a column containing salinized porous silica microspheres with a particle size of 5 μm and a 0.02 M HCl eluent.

19. The composition of claim 2, wherein for the at least one aluminum salt the peak 4 fraction of a size exclusion chromatography is from about 45% to about 70%, wherein the size exclusion chromatography uses a column containing salinized porous silica microspheres with a particle size of 5 μm and a 0.02 M HCl eluent.

20. The composition of claim 2, wherein for the at least one aluminum salt the peak 4 fraction of a size exclusion chromatography is from about 42% to about 75%, wherein the size exclusion chromatography uses a column containing salinized porous silica microspheres with a particle size of 5 μm and a 0.02 M HCl eluent.
